(19) United States
(12) Patent Application Publication
(10) Pub. No.: US 2011/0093050 A1
(43) Pub. Date: Apr. 21, 2011

(54) Insulated thermal therapy wrap designed specifically for podiatry

(76) Inventor: Elizabeth A. Damkoehler, Gulfport, FL (US)

(21) Appl. No.: 12/589,079

(22) Filed: Oct. 19, 2009

(51) Int. Cl. A61F 7/02

(52) U.S. Cl. 607/111; 607/114; 607/112

(57) ABSTRACT

The Footie is an insulated thermal wrap designed specifically for application to the foot, but can be used on other limbs such as ankles, knees and elbows as well. Noted unique features are that it is available in two sizes and a variety of colors and patterns. The interior layer of the wrap comprises an insulated pouch for containment of the thermal medium and can be used with either heat or cold pack therapy. The pouch itself is held closed by Velcro strips located at the top. The wrap encases the foot and is tapered at the top to fit the toe area. The wrap is held in place by two wide, stretchable Velcro straps which are strategically placed to allow for desired compression adjustability. The exterior layer of the wrap is made from flexible cloth. This is a low-cost treatment and allows the patient mobility while applying therapy for extended periods of time.

### REFERENCES CITED

U.S. Pat. No. 4,688,572 August 1987 Hubbard

U.S. Pat. No. 4,976,262 December 1990 Palmacci

U.S. Pat. No. 5,395,399 March 1995 Rosenwald

U.S. Pat. No. 4,055,188 October 1977 Pelton

U.S. Pat. No. 5,148,804 September 1992 Hill et al

### BACKGROUND OF THE INVENTION

It is well known that the application of heat or cold provides effective treatment of edema, muscle trauma; injuries such as sprains, contusions, strains, fractures, as well as post-surgical therapy for nueromas, bunionectomy, and sports related injuries.

The response to cryotherapy is improved if the cooling vehicle can be kept at a consistent temperature for longer periods of time. Unfortunately, most cooling vehicles lose their temperature in a relatively short period of time and must be replaced for extended treatment, which affects the patient's mobility. Additionally, in many applications, a controlled degree of compression on the affected area is desirable for enhancing the therapeutic effect as well.

The idea for this invention was created from a recent podiatry surgery where the post-surgical therapy recommended was consistent ice therapy. No product could be located, or obtained from the attending podiatrist who could ease the discomfort of trying to keep this area iced while also having some mobility. Even during the initial few days of being in a wheelchair, then on crutches, it was almost impossible to keep an icepack stationary on this appendage. Thermal wraps of different varieties were tried, some are disclosed in the following prior art. However, none were found that met the need for therapeutic effectiveness contoured to fit the foot combined with the ability to maintain the temperature for an extended period of time. Additionally, the custom sizing and fashion designs are not duplicated in any product that was researched by this inventor as well.

Prior art devices researched:

Hubbard patent U.S. Pat. No 4,688,572 granted Aug. 25, 1987 is a thermal pack for the knee.

Palmacci patent U.S. Pat. No.4,976,262 granted Dec. 11, 1990 is an ice bag for the knee.

Rosenwald patent U.S. Pat. No. 5,395,399 granted Mar. 7, 1995 is a thermal wrap for joints and limbs.

Pelton patent U.S. Pat. No. 4,055,188 granted Oct. 25, 1977 us a flexible bandage.

Hill et al. patent U.S. Pat. No. 5,148,804 granted Sept. 22, 1992 is a devise for applying cryotherapy.

### SUMMARY OF THE INVENTION

The invention provides an easy-to-use thermal wrap which allows for increased mobility. The mobility is increased by the ability to keep the foot elevated while applying therapy as well as the longevity of holding the temperature (up to 4 hours longer than those of the prior art). This therapeutic wrap is an insulated pouch which contains any readily available thermal medium (not customized or special ordered as in some prior art) held in place with a Velcro fastening system. The entire wrap is held in place over the affected appendage by two adjustable Velcro straps so the appropriate compression and location of the thermal medium can easily be adjusted.

The object is to provide a low-cost, aesthetically appealing, as well as functional treatment for either post-surgical treatment (such as bunion or neuroma removal) or general injuries (such as sprains, fractures, or contusions) of the human foot. A complete understanding of this invention may be obtained from the detailed description that follows along with the accompanying photographs.

### DESCRIPTION OF THE COLOR PHOTOGRAPHS

The file of this patent contains at least one photograph executed in color. Copies of this patent with color photographs will be provided by the Patent and Trademark Office upon request and payment of the necessary fee.

FIG. 1 is a photograph of the side view of the male version of the “Footie” on top shown with straps and with two sample patterns under it.

FIG. 2 is a photograph of the side view of the female version of the “Footie” on top shown with straps and with two sample patterns under it.

FIG. 3 shows the inner surface of the male Footie.

FIG. 4 shows the sealed Velcro strips being used which close the top of the thermal wrap and hold the thermal medium in place.

FIG. 5 shows a close-up side view of the male Footie with the stitched sides and the tapered top (top is narrower than bottom).

FIG. 6 shows the side view of a male wearing the Footie.

FIG. 7 shows the top view of a male wearing the Footie.

FIG. 8 shows the inner surface of the female Footie.

FIG. 9 shows the sealed Velcro strips beings used which close the top of the thermal wrap and hold the thermal medium in place.

FIG. 10 shows a close-up side view of the female Footie with the stitched sides and the tapered top top is narrower than bottom).

FIG. 11 shows the side view of a female wearing the Footie.

FIGS. 12 & 13 show two different top views of a female wearing the Footie.

### BEST MODE FOR CARRYING OUT THE INVENTION

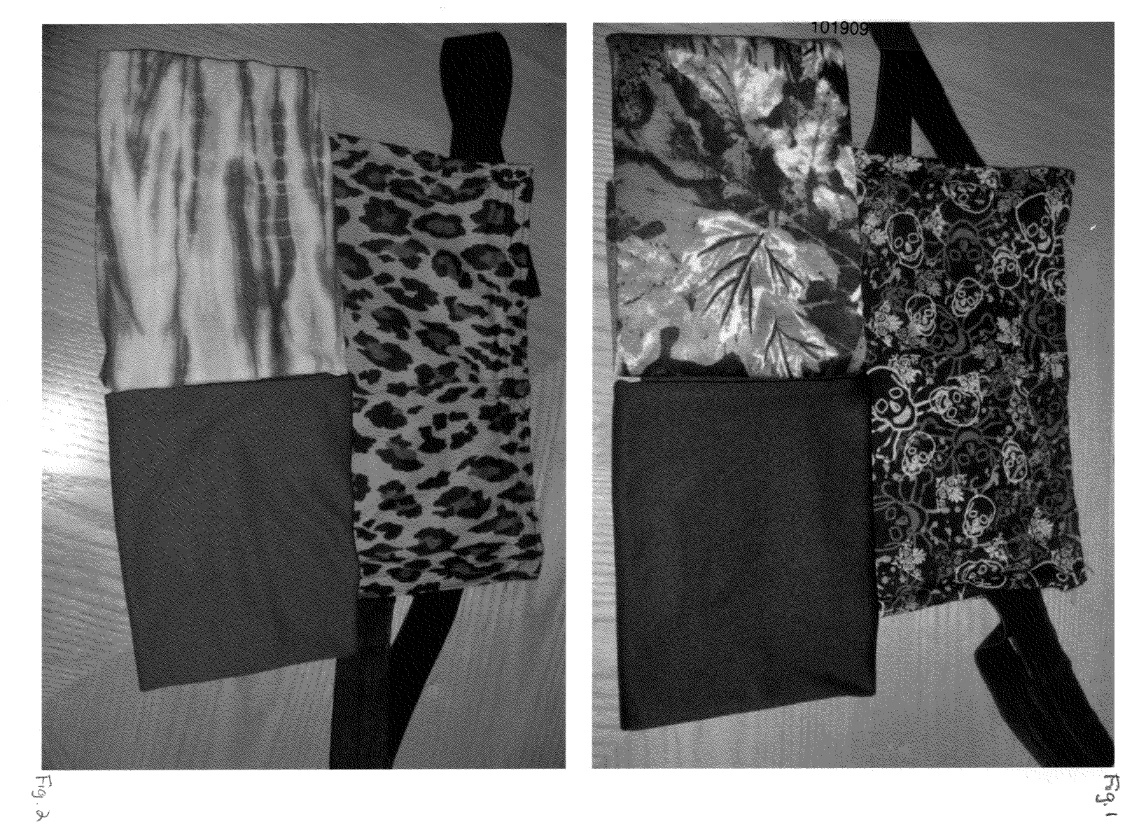

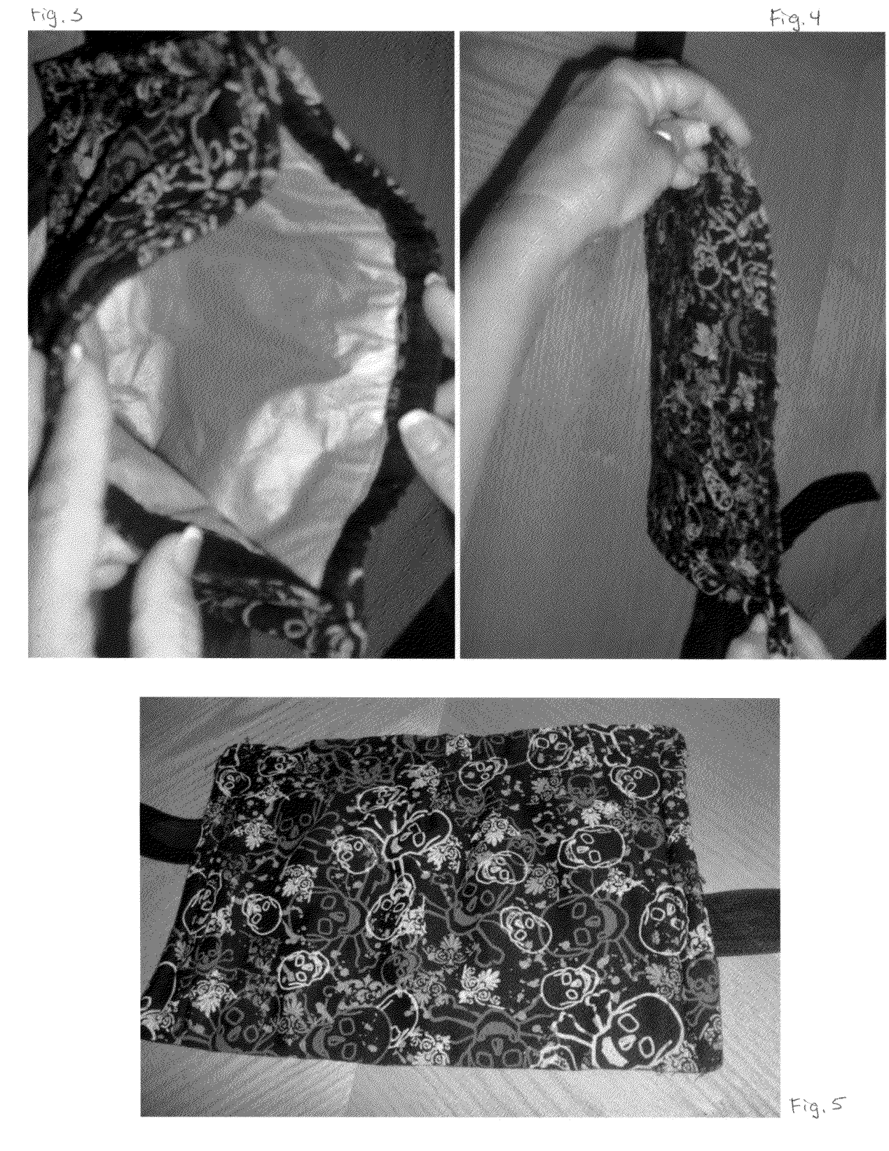

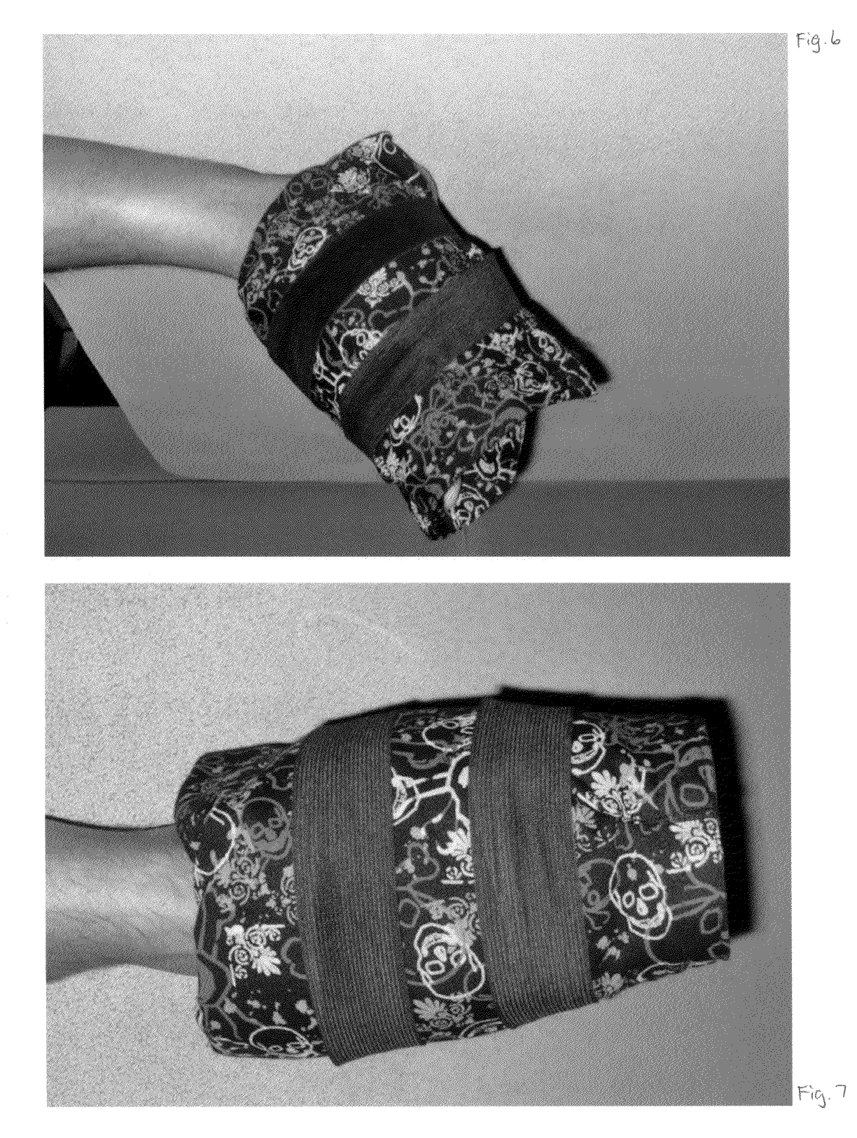

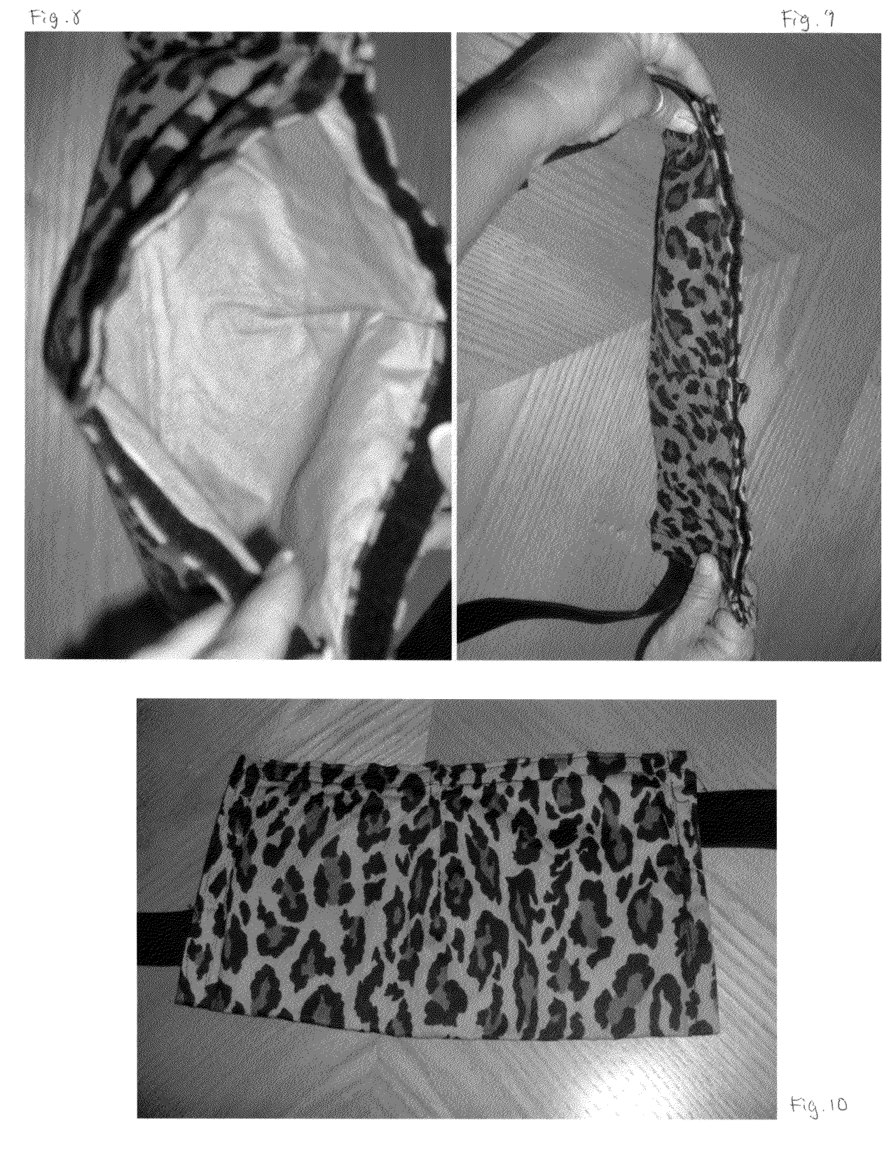

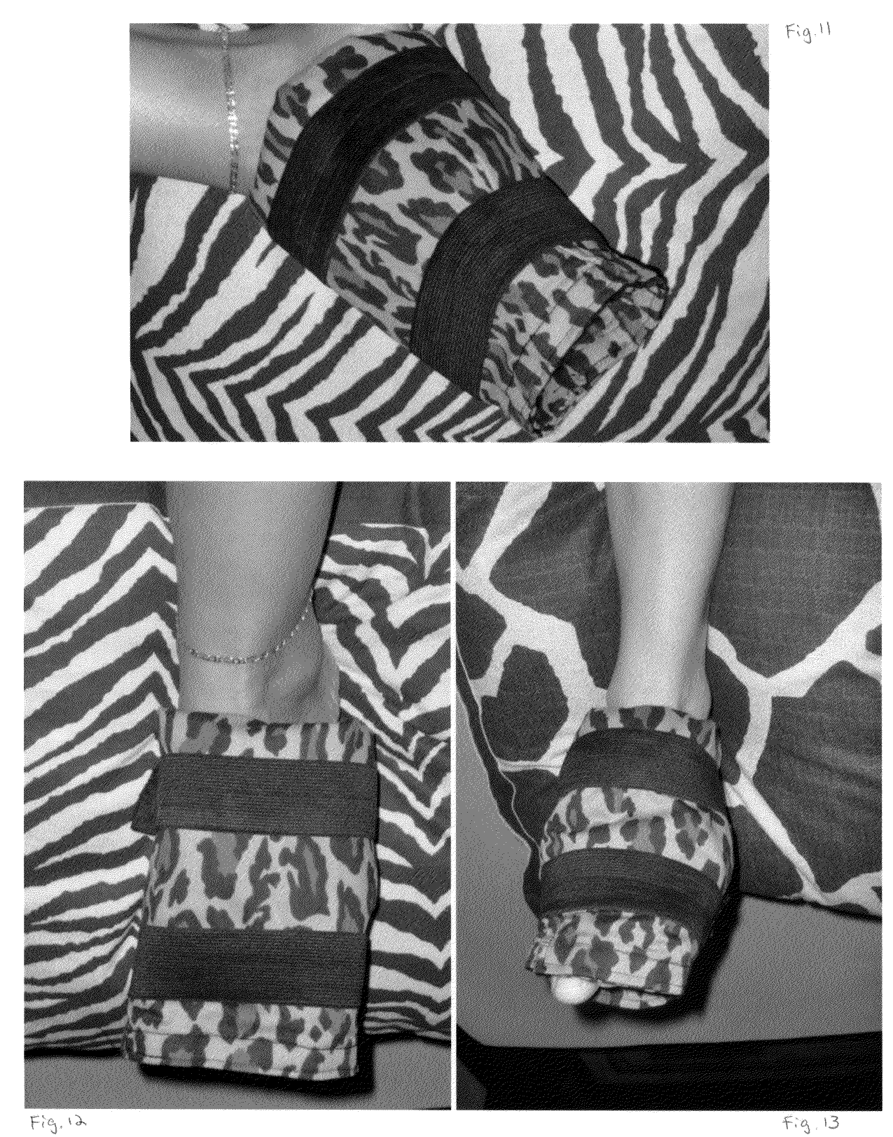

The present invention disclosed and claimed herein comprises a portable device, referred to here as “the Footie”, used to apply cryotherapy or heat therapy to the foot. As shown generally in FIGS. 3 and 8, the frozen or heat activated gel pack is inserted into the opening of the insulated pouch. The interior of the pouch is insulated with an aluminized coated fabric, which is similar to that used on an ironing board. The exterior of the wrap is made from a synthetic knit material which comes in a variety of colors and designs, is machine washable, durable, and flexible. The size of the pouch measures either 7×11.5 bottom) and 7×10.5 (top) for the women's, or 8.5×12 (bottom) and 8.5×11 (top) for the men's. Both are tapered at the top to form fit the foot. In the preferred embodiment, a frozen gel pack is placed into the pouch, which is then sealed with the Velcro strips along the top, as shown in FIGS. 4 and 9. The thermal wrap is wrapped around the foot in a single layer except for overlapping of the lateral edges by an amount determined by the size of the foot and the thickness of the ice pack used in the pouch. As shown in. FIGS. 5 and 10, a device structure is attached to the wrap structure on opposite sides. The device attachment comprises two stretchable elastic straps which are sewn into opposite sides of the wrap structure. The straps measure approximately 1.5 inches by 17 inches long and are located 2 inches from the top and bottom (respectively) of the wrap. Each strap has sewn into it a Velcro fastening system which consists of a section (3 inches×1.5 inches) on each side of each strap. Each strap contains one hook and one loop, sewn onto opposites of the strap. The hook side of the strap on the right side of the thermal wrap attaches to the loop side of the strap on the left of the thermal wrap, and vice versa. When looking at FIGS. 1 and 2, the upper side of each strap (on the left and on the right) has either a hook or loop sewn into it, and the bottom side has whatever the opposite would be sewn into that side. The length of the Velcro and the stretchable quality of the straps allow user to control the pressure of the wrap. A greater or lesser level of compression may be desired based on bandaging, suture pain, thickness of gel therapy being used, and expected level of activity.

### CONCLUSION

This present invention provides a single thermal wrap for the foot which obtains the advantages of support, mobility, compression, and cooling/heating depending on the prescribed therapy). It embodies a new structural arrangement and design which provide great improvement over the prior art thermal wraps. This invention has been used personally by the inventor for over 6 months with total satisfaction. The invention has been shown to and approved by local Podiatrists and Orthopedics as treatment for injuries or post-surgical treatment for the foot. It has been confirmed by said doctors that no other known product is available which is similar, and that they would recommend to their patients.

The cost of this product is low and should be readily available at pharmacies and similar retail businesses, not by prescription only. It can also be sold at Podiatrist offices. The wrap is easy to use and available in two sizes as well as a variety of colors and patterns. It can be sold with a gel pack, however standard gel packs are readily available.

1. A therapeutic thermal wrap comprising of:
a flexible elongated pouch fitted specifically for the foot, each side of the pouch having an outer wall and an inner, insulated wall.

2. A thermal wrap as defined in claim 1 where outer panels are of designer patterns and colors for aesthetic purposes, an inner wall made from aluminized material for insulation purposes. Said inner lining prevents dampness and condensation on the outside, while allowing cold (or heat) to get to the injured area being treated. Insulated inner lining holds gel pack temperature for up to 4 hours longer than similar prior art tested.

3. A therapeutic wrap as defined in claim 2 wherein said insulated pocket is receivable for a standard refrigerant gel pack, or heated gel pack, of various sizes (up to 6×10.5).

4. A therapeutic wrap as defined in claim 3 wherein said pocket is open-able from the top, connected at the bottom, and stitched on two sides. The opening at the top is held together with two Velcro strips (one hook side and one loop side).

5. A thermal wrap for the foot as defined in claim 1 which has stretchable straps sewn into each side that wrap around the foot and is attached with Velcro strips for closure. Said straps are adjustable for desired pressure and degree of activity, one strap is located for tension at the forefoot, and the other is located for tension just in front of the heel.

6. A therapeutic thermal wrap is claimed which is inexpensive, can be used with standard gel packs, is available in men's and women's sizes, and comes in a variety of colors as well as a choice of designs and patterns.
